(19) United States
(12) Patent Application Publication
(10) Pub. No.: US 2025/0314630 A1
(43) Pub. Date: Oct. 9, 2025

(54) LIPID BILAYERS WITH HOLEY GRIDS

(71) Applicant: The University of Chicago, Chicago, IL (US)

(72) Inventor: Vishal Maingi, Chicago, IL (US)

(21) Appl. No.: 19/083,105

(22) Filed: Mar. 18, 2025

Related U.S. Application Data
(60) Provisional application No. 63/566,800, filed on Mar. 18, 2024.

(51) Int. Cl. G01N 33/00; B82Y 15/00; G01N 23/2251

(52) U.S. Cl. CPC G01N 33/0096; G01N 23/2251; B82Y 15/00; G01N 2405/00

(57) ABSTRACT

Provided herein are porous, thin substrates (e.g., holey grids) comprising lipid bilayers formed across and/or within holes in the substrates, methods of forming bilayers within the holes of porous, thin substrates (e.g., holey grids), and methods of conducting structural analysis (e.g., by cryoEM) of membrane proteins, membrane-embedded particles, or membrane-attached particles within the lipid bilayers.

### CROSS REFERENCE TO RELATED APPLICATIONS

This application claims priority to and the benefit of U.S. Provisional Patent Application No. 63/566,800, filed Mar. 18, 2024, which is incorporated herein by reference in its entirety and for all purposes.

### FIELD

Provided herein are porous, thin substrates (e.g., holey grids) comprising lipid bilayers formed across and/or within holes in the substrates, methods of forming bilayers within the holes of porous, thin substrates (e.g., holey grids), and methods of conducting structural analysis (e.g., by cryoEM) of membrane proteins, membrane-embedded particles, or membrane-attached particles within the lipid bilayers.

### BACKGROUND

Cryo-electron microscopy (cryoEM) has paved the way for structure determination of many membrane proteins (and also the soluble proteins) without the need for crystallisation. Solving the structure of membrane proteins and their complexes is important to understand their function and their mechanistic role in pathogenesis, and also useful for the drug discovery process. To weigh in their importance, more than 60% of the targets of medicinal compounds in the market are membrane proteins and now pharmaceutical companies are establishing their own cryoEM facilities which was earlier limited to pay-for-a-service or academic collaborations. However, for many membrane proteins it is difficult to determine their structure in native lipid bilayers or near native lipid environment. Thus, developing a generic method to solve membrane protein structures, either individually or in complex, within their native lipid membrane environment or near native lipid environment would eventually have a large impact on human health.

### SUMMARY

Provided herein are porous, thin substrates (e.g., holey grids) comprising lipid bilayers formed across and/or within holes in the substrates, methods of forming bilayers within the holes of porous, thin substrates (e.g., holey grids), and methods of conducting structural analysis (e.g., by cryoEM) of membrane proteins, membrane-embedded particles, or membrane-attached particles within the lipid bilayers.

In some embodiments, provided herein are compositions comprising: (a) a substrate having a hydrophobic surface; (b) a plurality of apertures through the substrate material; and (c) a lipid bilayer extending across one or more of the plurality of apertures. In some embodiments the shape of aperture is circular, oval, square, rectangle, irregular, symmetric or asymmetric polygonal or any geometry shape etc.

In some embodiments, the substrate is hydrophobic. In some embodiments, the substrate comprises a material is selected from carbon, polytetrafluoroethylene (PTFE [TEFLON]), polydimethylsiloxane, fluorinated ethylene propylene (FEP), fluorosilane, octadecyltrichlorosilane (OTS), polyethylene, perfluoroalkoxy (PFA), and gold (chemically modified). In some embodiments, the substrate material is a carbon material selected from an amorphous carbon film, pyrolytic carbon film, graphene, graphene oxide, and carbon nanotubes. In some embodiments, the substrate material is a chemically-modifiable material and has been modified to display a hydrophobic surface group. In some embodiments, the chemically-modifiable material is selected from silicon dioxide (SiO₂), titanium dioxide (TiO₂), aluminum oxide (Al₂O₃), zirconium oxide (ZrO₂), polyethylene (PE), polydimethylsiloxane (PDMS), a fluoropolymer (e.g., PTFE, FEP, PFA), polypropylene (PP), polytetrafluoroethylene (PTFE), and polyvinylidene fluoride (PVDF). In some embodiments, the hydrophobic surface group is selected from lipids, sterols, hydrocarbon chains, fatty acids, synthetic polymers, nanoparticles, short-chain perfluorocarbons, chemically modified polyethylene glycol or any hydrophobic chemistry. In some embodiments, the substrate has a thickness of 1-100 nm (e.g., 1 nm, 2 nm, 3 nm, 4 nm, 5 nm, 10 nm, 15 nm, 20 nm, 25 nm, 30 nm, 40 nm, 50 nm, 100 nm or ranges therebetween). In some embodiments, the thickness of the substrate tapers at the edge of the apertures. In some embodiments, the substrate has a thickness at an edge of the apertures of 1 to <100 nm (e.g., 1 nm, 2 nm, 3 nm, 4 nm, 5 nm, 6 nm, 7 nm, 8 nm, 9 nm, 10 nm, 12 nm, 15 nm, <100 nm, or ranges therebetween). In some embodiments, the plurality of apertures have dimensions (e.g., length width, diameter, etc) between 50 nm and 20 μm (e.g., 50 nm, 100 nm, 200 nm, 300 nm, 400 nm, 500 nm, 1 μm, 2 μm, 3 μm, 4 μm, 20 μm or ranges therebetween (e.g., between 300 nm and 1 μm)). In some embodiments, the apertures are separated from each other by 50 nm to 20 μm (e.g., 50 nm, 100 nm, 200 nm, 300 nm, 400 nm, 500 nm, 1 μm, 2 μm, 3 μm, 4 μm, 20 μm or ranges therebetween (e.g., between 300 nm and 1 μm)). In some embodiments, the lipid bilayer encompasses the substrate material at the edge of the one or more apertures.

In some embodiments, the lipid bilayer comprises one or more components selected from glycerophospholipids, sphingolipids, glycolipids, sterols, fatty acids, diacylglycerol, ether lipids, natural or synthetic lipidome, cell membrane extracted lipids mix, lipids with modified chains and/or headgroups, fluorescent lipids, PEGylated lipids or any chemical modifications on these components. In some embodiments, the lipid bilayer further comprises one or more molecular or macromolecular structures of interest. In some embodiments, the one or more molecular or macromolecular structures of interest are selected from membrane proteins, membrane embedded/associated particles, hydrophobic DNA, DNA nanostructures, peptides, and polymers. In some embodiments, the membrane proteins are selected from transmembrane proteins, integral membrane proteins, peripheral membrane proteins, lipid-anchored proteins, and any complex formed by these components with or without soluble proteins or DNA or nanoparticles or biomolecules or ligands or drugs.

In some embodiments, compositions herein further comprise a mesh support beneath the substrate material. In some embodiments, the mesh support comprises a material selected from copper, gold, nickel, rhodium, molybdenum, silicon, carbon, metal alloys or any metal. In some embodiments, the mesh support comprises a plurality of interwoven or crossed metallic bands with gaps between them, where the bands are 1-200 μm in width (e.g., 1 μm, 2 μm, 5 μm, 10 μm, 20 μm, 50 μm, 100 μm, 200 μm and ranges therebetween) and the gaps are 25-400 μm wide (e.g., 25 μm, 50 μm, 75 μm, 100 μm, 150 μm, 200 μm, 250 μm, and ranges therebetween). In some embodiments, the mesh support comprises an outer rim where the rim width is 50-800 μm (e.g., 50 μm, 75 μm, 100 μm, 150 μm, 200 μm, 250 μm, 800 μm and ranges therebetween).

In some embodiments, provided herein are methods of preparing a composition described herein, the methods comprising: (a) combining (i) components of the lipid bilayer, (ii) one or more detergents, and (iii) the substrate; and (b) removing the one or more detergents under conditions that allow for formation of the lipid bilayer across one or more of the plurality of apertures. In some embodiments, the one or more detergents are removed over a period of time ranging from 5 minutes to 48 hours (5 minutes, 10 minutes, 20 minutes, 30 minutes, 45 minutes, 1 hour, 2 hours, 4 hours, 6 hours, 12 hours, 24 hours, 48 hours, or more or ranges therebetween).

In some embodiments, (i) the components of the lipid bilayer, (ii) the one or more detergents, and (iii) the substrate are combined in a vessel that is permeable to the one or more detergents. In some embodiments, the vessel comprises one or more surfaces that are a dialysis membrane. In some embodiments, the one or more detergents are removed by placing the vessel in a solution or mixture with a lower concentration of the one or more detergents than within the vessel, and allowing the detergents to dialyze out of the vessel. In some embodiments, the solution or mixture is detergent frec.

In some embodiments, the one or more detergents are removed adding an adsorbing agent capable of adsorbing the one or more detergents. In some embodiments, the adsorbing agent comprises one or more of polystyrene, polyethylene glycol, polysorbate, polyvinyl alcohol, poly(ethyleneimine), sepharose, silica resins, ion exchange resins, activated charcoal, fullerene derivatives, cyclodextrins, silica gel, alumina, and bovine serum albumin.

In some embodiments, methods further comprise a step before step (b) of adding one or more molecular or macromolecular structures of interest selected from membrane proteins, membrane embedded/associated particles, hydrophobic DNA, DNA nanostructures, peptides, polymers, ligands, drugs, soluble proteins. In some embodiments, the membrane proteins are selected from transmembrane proteins, integral membrane proteins, peripheral membrane proteins, and lipid-anchored proteins.

In some embodiments, methods further comprise a step after step (b) of contacting the composition with one or more molecular or macromolecular structures of interest within a lipid-containing complex and allowing the one or more molecular or macromolecular structures of interest to transfer from the lipid-containing complex to the lipid bilayer extending across one or more of the plurality of apertures. In some embodiments, the lipid-containing complex is selected from nanodiscs, liposomes, micelles, lipid rafts, lipoplexes, lipid-polymer complexes, and proteoliposomes. In some embodiments, the lipid bilayer already has one or more molecular or macromolecular structures of interest selected from membrane proteins, membrane embedded/associated particles, hydrophobic DNA, DNA nanostructures, peptides, polymers, ligands, drugs, soluble proteins.

In some embodiments, provided herein are methods of preparing a composition described herein, comprising: (a) applying lipids with or without additional macromolecules in an organic solvent to the substrate; (b) drying the organic solvent; (c) adding an aqueous buffer solution, thereby establishing a lipid bilayer extending across one or more of the plurality of apertures.

In some embodiments, provided herein are methods of preparing a composition described herein, comprising: (a) forming a lipid monolayer at a water-air interface with or without additional macromolecules; (b) raising the substrate through the water-air interface; (c) raising the substrate above the water-air interface, thereby forming the lipid bilayer extending across one or more of the plurality of apertures.

In some embodiments, methods provided herein further comprise contacting the composition with one or more molecular or macromolecular structures of interest within a lipid-containing complex and allowing the one or more molecular or macromolecular structures of interest to transfer from the lipid-containing complex to the lipid bilayer extending across one or more of the plurality of apertures. In some embodiments, the lipid-containing complex is selected from liposomes, micelles, lipid rafts, lipoplexes, lipid-polymer complexes, and proteoliposomes. In some embodiments, the lipid bilayer already has one or more molecular or macromolecular structures of interest selected from membrane proteins, membrane embedded/associated particles, hydrophobic DNA, DNA nanostructures, peptides, polymers, ligands, drugs, soluble proteins.

In some embodiments, provided herein are methods of analyzing the structure of a molecular or macromolecular structures of interest embedded and/or associated with the lipid bilayer of the composition described herein, the method comprising conducting cryoEM analysis lipid bilayer extending across one or more of the plurality of apertures.

In some embodiments, any of the compositions, components, methods, and method steps described herein may be combined in any suitable manner within the scope herein.

### BRIEF DESCRIPTION OF THE DRAWINGS

FIG. 1. A full-size EM grid is shown on the left and a zoom-in shows a substrate layer with holes above the mesh support.

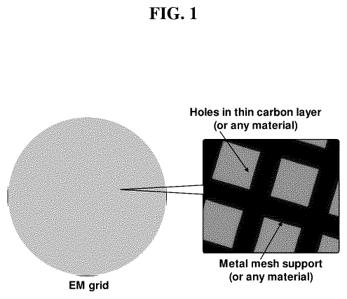

FIG. 2. Lipid assembly in holey grids using dialysis approach.

FIG. 3. EM grid 1.2 μm diameter holes seen with 20× objective using Evos M5000 inverted microscope.

FIG. 4. Free-standing lipid bilayer formed in tapered holes and other possible tapered shape types also shown.

FIG. 5. Cartoon showing the elements that will be used to model the carbon hole with free-standing lipid bilayer at the coarse-grained level (with lipids, carbon layer, water & ions); water, ions are not shown.

### DETAILED DESCRIPTION

Provided herein are porous, thin substrates (e.g., holey grids) comprising lipid bilayers formed across and/or within holes in the substrates, methods of forming bilayers within the holes of porous, thin substrates (e.g., holey grids), and methods of conducting structural analysis (e.g., by cryoEM) of membrane proteins, membrane-embedded particles, or membrane-attached particles within the lipid bilayers.

As used herein, the term “holey grid” in general refers to a substrate typically used in microscopy, particularly in cryo-EM, comprising a thin substrates layer of an electron-transparent material, such as carbon, gold, or other suitable materials, which has a plurality of apertures distributed across the surface. These apertures allow for the transmission of electrons through the grid and facilitate the observation of a sample placed on the grid, typically by providing regions where the sample can be viewed with minimal interference from the grid material. The apertures in a holey grid can vary in size and spacing, typically ranging from approximately 50 nm to 25 μm in diameter, and are arranged to optimize the balance between grid support for the sample and minimal scattering of electrons, thereby improving imaging quality in high-resolution microscopy techniques. In some embodiments, the compositions described herein are hole grids having lipid bilayers formed in the apertures thereof.

Cryo-electron microscopy (cryo-EM) is a powerful imaging technique used to observe biological molecules at high resolution in a near-native, frozen state. Unlike traditional electron microscopy, which requires samples to be fixed onto a surface and often stained with heavy metals, cryo-EM allows for the examination of samples that are rapidly frozen, typically in liquid ethane, preserving their natural structure without the need for staining, dehydration, or other sample manipulation. In a typical cryoEM protocol, a thin layer of a sample (e.g., protein, macromolecular complex, virus, or other macromolecule) is placed on a grid, blotted to remove extra solvent and rapidly frozen (e.g., in liquid ethane), creating vitreous ice and avoiding damage to the biological sample that might occur from ice crystallization. The sample is then imaged using an electron microscope. Since the sample is frozen in its natural state but in different orientations, it appears as a 2D projection of the 3D structure. In contrast, for cryo-electron tomography (cryo-ET) multiple images of the sample are taken from different angles as tilt-series. These 2D projections obtained from different orientations and/or angles are processed and combined using advanced computational methods to generate a 3D reconstruction of the sample. Cryo-EM has revolutionized structural biology, as it allows for the visualization of large and complex biomolecular structures, such as membrane proteins and molecular machines, that are difficult to study using traditional methods like X-ray crystallography or nuclear magnetic resonance (NMR) spectroscopy.

CryoEM technology has revolutionized structure determination of proteins without the need for crystallization, a major requirement for X-ray diffraction studies. Since 1990, when the first high-resolution protein structure was reported using cryoEM, significant developments in sample preparation, hardware, and software have facilitated the determination of over 10,000 protein structures using this technique. It is expected that cryoEM will surpass the total number of structures solved using X-ray crystallography. For cryoEM only a few microliters of sample is required, which is loaded onto a grid and plunge-frozen in liquid ethane, or a mixture of ethane with propane, after excess solvent is removed using a blotting method. This process results in a thin layer of amorphous ice with protein molecules ‘fixed’ in random orientations. The frozen grids are then imaged using a transmission electron microscope. Subsequently, single-particle analysis (SPA), involving many projections of multiple copies of the same protein in different orientations, or sub-tomogram averaging (STA), which entails collecting projections for multiple copies of protein molecules as tilt-angle series, can be used to reconstruct the cryoEM map and build atomic models.

Unlike soluble proteins, which are in general stable in aqueous buffer solution, maintaining the structural and functional stability of purified membrane proteins is challenging. The hydrophobic domains of membrane proteins require stabilization in an aqueous carrier solution using, for example, detergents, amphipols, or nanodiscs. Each of the stabilizing techniques for membrane proteins comes with its own set of advantages and disadvantages. Despite these challenges, it is noteworthy that even for membrane proteins, de novo atomic models are achievable using ˜3.5 Å cryoEM maps. Membrane proteins are typically extracted and purified in buffer solutions containing detergents or amphipols to prevent aggregation and precipitation, facilitating their grid preparation for cryoEM. However, this method has significant drawbacks: detergents or amphipols don't provide a native lipid environment for membrane proteins, screening various detergents is often necessary to maintain protein structure and function, the native lipids interacting with hydrophobic domains are often lost in the presence of detergents. Alternatively, another approach is to use nanodiscs to host a membrane protein embedded in the lipid bilayer. Nanodiscs which are formed by membrane scaffold protein (MSP) is becoming a method of choice to prepare membrane protein samples for cryoEM studies. In brief, to prepare a nanodisc a purified protein in a detergent solution is mixed with desired lipids and a MSP, and after the detergent is removed two MSP belts surround a planar lipid bilayer patch holding a membrane protein inside. This approach allows a native or near-native lipid environment around the membrane protein and depending upon the size and shape of the protein one can choose a different size of MSP which also defines the overall diameter of a nanodisc. However, using nanodiscs involves additional purification steps, potential membrane protein-MSP interactions, and issues with heterogeneity in size and shape, which can interfere with cryoEM structure analysis. One simplified approach is to form proteoliposomes by mixing purified membrane proteins in a detergent with lipids and dialysing out the detergent. Main disadvantage is that large size of the proteoliposomes can result in thick ice (detrimental for cryoEM) and also the curvature in the liposome can affect the structure properties of the membrane proteins. The relatively new technique known as microcrystal electron diffraction (MicroED) combines both X-ray diffraction and cryoEM methods to diffract protein nanocrystals. MicroED has been reported to solve the structure of membrane protein (e.g., voltage dependent ion channel) by mixing the protein with bicelles and later growing them as nanocrystals. However, this process resulted in a viscous sample and posed difficulties for the grid preparation. Also, MicroED has been used to solve G-protein coupled receptors (GPCR) structure obtained from the crystals grown in lipid cubic phase (LCP). The gel type nature of the LCP presents a challenge for grid preparation and to overcome that it was converted to sponge liquid like phase. Nevertheless, main disadvantages with MicroED are difficulty in preparing and identifying nanocrystals suitable for MicroED, and the necessity to mill nanocrystal into thin lamellac using a focused ion beam adds an extra essential step to the process.

CryoEM allows for resolving the structures in a lipid environment and different methods have been developed for sample preparation and analyses suitable for single-particle analyses (SPA) or subtomogram averaging (STA). For sample preparation for cryoEM, membrane proteins can be stabilized, for example, by using suitable detergents, by formation of proteoliposomes (membrane proteins embedded in liposome bilayer), or by using amphiphilic polymer or protein based scaffolds forming nanodiscs (membrane proteins hosted by scaffolded planar lipid bilayer). Once prepared, the samples can be applied on glow discharged holey grids (e.g., a perforated carbon layer supported on copper or gold mesh) for SPA/STA. Different sample preparation methodologies have pros and cons. For instance, detergents don't provide native lipid environment, scaffolded nanodiscs are heterogenous in terms of size and/or hosting more than one protein per nanodisc, which can be problematic for downstream cryoEM SPA, and many of these procedures require additional time-consuming purification steps which can be detrimental for the membrane proteins (e.g., in terms of their structure and functional stability).

To overcome the issues present in the field, and to provide a simplified approach, provided herein are methods for the assembly of free-standing lipid bilayers in nanoscale to microscale apertures in solid substrates. Provided herein are holey grids and similar devices comprising a lipid bilayer extending across the apertures of the grid. The full scope of embodiments herein is not limited by the types of holey grids or other substrates that the present technology finds use with. Although various materials, arrangements, and dimensions are described herein for holey grids and related devices, the full breadth of the present technology is not limited by the type of holey grid and/or the characteristics of the apertures across which lipid bilayers are assembled and/or extend.

Cryo-EM grids are used to support the biological samples for imaging. These grids are typically small (e.g., 3.05 mm or 2.03 mm in diameter), flat, metal (e.g., copper, nickel, gold, molybdenum, titanium, stainless steel, aluminum etc.) meshes (e.g., 100-1000 squares per inch (e.g., 200 mesh, 400 mesh, etc.)) with a thin (e.g., 2-50 nm) film of substrate material (e.g., carbon (e.g., amorphous carbon film, pyrolytic carbon film, graphene, graphene oxide, and carbon nanotubes), polytetrafluoroethylene (PTFE [TEFLON]), polydimethylsiloxane, fluorinated ethylene propylene (FEP), fluorosilane, octadecyltrichlorosilane (OTS), polyethylene, perfluoroalkoxy (PFA), gold, etc.) extending over the mesh support (e.g., on top of the mesh, covering one side of the grid, etc.). In general cryoEM applications, grids may be solid/continuous grids (e.g., the substrate material forms a continuous film that spans the entire grid; solid/continuous films are useful for certain types of samples, but can create background noise and interfere with imaging because the electron beam interacts with the entire grid area) or holey grids (e.g., comprising an array of apertures in which sample can be placed for analysis). Embodiments described herein find particular use with holey grids.

In some embodiments, a holey grid minimally comprises a substrate material (e.g., carbon) with an array of apertures therethrough. The apertures may be regularly or irregularly spaced across the substrate and may have any suitable spacing, dimensions, shape, geometry and/or arrangement. In typical embodiments, the substrate rests or is adhered to one side of a mesh (e.g., metal) support. Each opening (e.g., square, triangle, polygon, etc.) in the mesh provides a planar expanse of the substrate through which one or more (e.g., 1, 2, 3, 4, 5, 10, 20, 50, 100, or more) apertures reside. Provided herein are methods for assembling a lipid bilayer across the apertures of a holey grid, holey grids comprising lipid-bilayers spanning the apertures therein, methods of placing/including macromolecules and molecular complexes of interest into such lipid-bilayer-containing apertures, and methods of using such holey grids for structural analysis of macromolecules and molecular complexes of interest.

Existing holey grids that may find use in embodiments herein (e.g., for assembly of lipid bilayers across the apertures thereof) include QUANTIFOIL grids, ULTRAUFOIL grids, C-flat grids and lacey grids. While grids can be customized in laboratory settings, they are also readily available from commercial suppliers. Among the most frequently utilized commercial holey carbon grids are the ‘Quantifoil’ and ‘C-flat’ types, characterized by varying carbon thicknesses of 10-40 nm and hole diameters ranging from 0.6 to 17 μm. Notably, hole sizes of 1.2 μm and 2 μm are predominantly utilized for single-particle analysis (SPA) and sub-tomogram averaging (STA), respectively. In existing techniques, some steps of which may find use in embodiments herein, prior to loading the sample, the hydrophobic carbon grids undergo glow discharge treatment to render them hydrophilic, facilitating the uniform dispersion of the aqueous sample. For membrane proteins, 2-4 μL of the sample, prepared with detergents, nanodiscs/polymers, or proteoliposomes, is loaded onto the glow-discharged grids. Subsequently, the grid bearing the sample is blotted with filter paper to remove the excess solvent before plunge freezing in liquid ethane.

Provided herein are holey grids comprising a substrate (a surface material or materials through which lipid-bilayer-containing apertures reside). In some embodiments, the substrate is hydrophobic, has a hydrophobic surface, is coated in a hydrophobic material or agent, and/or is surface modified to present a hydrophobic surface.

In some embodiments, the substrate comprises a hydrophobic material, for example, a material selected from carbon, silicon nitride (Si₃N₄), polydimethylsiloxane (PDMS), polytetrafluoroethylene (PTFE [TEFLON]), polydimethylsiloxane, fluorinated ethylene propylene (FEP), fluorosilane, octadecyltrichlorosilane (OTS), polyethylene, perfluoroalkoxy (PFA), and gold. In some embodiments, the substrate material is a carbon material selected from an amorphous carbon film, pyrolytic carbon film, graphene, graphene oxide, carbon nanotubes, etc.

In some embodiments, the substrate comprises a chemically-modifiable material and has been modified to display a hydrophobic surface group. In some embodiments, the chemically-modifiable material is selected from silicon dioxide (SiO₂), titanium dioxide (TiO₂), aluminum oxide (Al₂O₃), zirconium oxide (ZrO₂), polyethylene (PE), polydimethylsiloxane (PDMS), a fluoropolymer (e.g., PTFE, FEP, PFA), polypropylene (PP), polytetrafluoroethylene (PTFE), and polyvinylidene fluoride (PVDF). In some embodiments, exemplary hydrophobic surface groups for hydrophobic modification of surface groups include lipids, sterols, hydrocarbon chains, fatty acids, synthetic polymers, nanoparticles, short-chain perfluorocarbons, chemically modified polyethylene glycol or any hydrophobic chemistry. In some embodiments, a substrate surface is modified to display a hydrocarbon-based hydrophobic group, such as an aliphatic hydrocarbon (e.g., alkly chain (e.g., methyl, ethyl propyl, butyl, octyl, decyl, or longer)), a branched alkyl group (e.g., iso-butyl, iso-octyl, neo-pentyl, etc.), cycloalkyl groups, aromatic hydrocarbons, etc.), etc. In some embodiments, a substrate surface is modified to display a fluorocarbon-based hydrophobic group, such as a fluorinated alkyl (e.g., fluoromethyl (—CF₃), perfluoromethyl (—CF₂CH₃), perfluorohexyl (—C₆F₁₃), perfluorooctyl (—C₈F₁₇), Perfluorodecyl (—C₁₀F₂₁), etc.), perfluorinated aromatic groups (e.g., Perfluorobenzene, Perfluorotoluene, etc.), fluoropolymer groups (e.g., polytetrafluoroethylene (PTFE), fluoroethylenepropylene (FEP), etc.), etc. In some embodiments, a substrate surface is modified to display a long-chain fatty acid, such as a saturated fatty acid (e.g., lauric acid (C₁₂H₂₃COOH), stearic acid (C₁₈H₃₅COOH), palmitic acid (C₁₆H₃₁COOH), arachidic acid (C₁₉H₃₉COOH), etc.) or unsaturated fatty acid (e.g., oleic acid (C₁₈H₃₄COOH) (monounsaturated), linoleic acid (C₁₈H₃₂O₂) (polyunsaturated), alpha-linolenic acid (C₁₈H₃₀O₂) (polyunsaturated), etc.). In some embodiments, a substrate surface is modified to display a hydrophobic lipid and/or membrane-associated molecule, such as phospholipid groups (e.g., phosphatidylcholine (PC), phosphatidylethanolamine (PE), phosphatidylserine (PS), etc.), cholesterol and steroid groups (e.g., cholesterol (C₂₇H₄₆O), cholic acid (C₂₄H₄₀O₄), etc.), glycolipid groups (e.g., cerebrosides, gangliosides, etc.), etc. In some embodiments, a substrate surface is modified to display a polymeric hydrophobic group, such as polyethylene (PE) groups (e.g., polyethylene (C₂H₄)ₙ), low-density polyethylene (LDPE), etc.), polystyrene (PS) groups (e.g., polystyrene (C₈H₈)ₙ), polypropylene (PP) groups (e.g., polypropylene (C₃H₆)ₙ), polyvinyl chloride (PVC) groups (e.g., polyvinyl chloride (C₂H₃Cl)ₙ), etc. In some embodiments, a substrate surface is modified to display a hydrophobic biopolymer, such, as a peptide comprising hydrophobic amino acids (e.g., Ala, Val, Leu, Ile, Phe, Trp, Met).

Hydrophobic modifications of coatings to a substrate material may be made by any suitable chemistry and may involve covalent and/or non-covalent interactions. Whether a hydrophobic substrate material is used, a substrate material is chemically modified to present a hydrophobic surface, or a substrate material is coated to present a hydrophobic surface, substrates for use in certain embodiments herein present a hydrophobic surface suitable for interfacing with a lipid monolayer or bilayer assembled over the substrate and/or a lipid bilayer assembled within/across the apertures within the substrate.

In some embodiments, a substrate may comprise a single layer of material or multiple (e.g., 2, 3, 4, or more) layers. The layers may be of the same or different materials. The different layers (materials) may impart different characteristics to the substate (e.g., surface hydrophobicity, adherence to the mesh, rigidity, flexibility, electron transparency, etc. In some embodiments, a suitable substrate may comprise any combination of materials, coatings, surface modifications, etc. described herein.

In some embodiments, a substrate has a thickness of 1-100 nm (e.g., 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 20 nm, 25 nm, 30 nm, 35 nm, 40 nm, 45 nm, 50 nm, 100 nm or ranges or thicknesses therebetween). A substrate may have a consistent thickness throughout (e.g., within 10%, 5%, 1%, etc.) or may comprise regions of thicker or thinner substrate. A substrate may tapper (e.g., reduced thickness) in the region immediately surrounding an aperture. For example, a substrate may be >15 nm thick (e.g., 16 nm, 20 nm, 25 nm, 30 nm, or more) but tapper to <13 nm thick (e.g., 12 nm, 10 nm, 9 nm, 8 nm, 7 nm, 6 nm, 5 nm, 4 nm, 3 nm, 2 nm) in the immediately surrounding an aperture (e.g., tapering beginning at 5-20 nm from the circumference of the aperture).

The grids provided herein comprise a substrate with one or more apertures therethrough. Typically, the grids comprise many (e.g., 20, 50, 100, 200, 500, 1000, or more) apertures across the entire grid. The apertures may be circular, oval, approximately circular, spherical, polygonal (e.g., square), rectangular (e.g., a slit), or any suitable shape. The apertures may be regularly spaced over the substrate, irregularly spaced, or otherwise arranged. For example, apertures may be regularly spaced or otherwise patterned over the opening in the mesh beneath the substrate. In some embodiments, each mesh opening houses the same (or approximately the same) number of apertures through the substrate (e.g., 1, 2, 3, 4, 5, 10, 20, 50, or more). The apertures in a mesh opening may be arranged in any pattern (e.g., a grid) or organization, including random distribution. In some embodiments, the apertures through the substrate are generated by any suitable method, including radiations, mechanically (e.g., puncturing or cutting a perforation through the substrate), chemically (e.g., using a chemical reaction to eat away portions of the substrate), enzymatically, synthetically (e.g., the substrate is synthesized with the apertures in place), using soft lithography nanofabrication approach with poly(dimethylsiloxane) stamps etc.

In some embodiments, apertures have dimensions (e.g., diameter, width, length, thickness, height) of between 1 nm and 25 μm (e.g., 1 nm, 3 nm, 4 nm, 5 nm, 6 nm, 7 nm, 8 nm, 9 nm, 10 nm, 12 nm, 14 nm, 16 nm, 18 nm, 20 nm, 25 nm, 30 nm, 35 nm, 40 nm, 45 nm, 50 nm, 25 μm or ranges therebetween). All apertures of a grid may have the same dimensions (e.g., within 10%, 5%, 2%, 1%, etc.) or may be of varying sizes. In some embodiments, apertures are separated from adjacent apertures by at least 50 nm to 20 μm (e.g., 50 nm, 100 nm, 200 nm, 300 nm, 400 nm, 500 nm, 750 nm, 1 μm, 2 μm, 3 μm, 4 μm, 20 μm or ranges therebetween).

As described elsewhere herein, a grid may comprise a mesh support beneath the substrate. In some embodiments, a mesh support comprises a series of interwoven and/or intersecting wires or bands with spaces between the wires/bands. Depending upon the pattern and spacing of the wires/bands, the spaces within the mesh may be circular, oval, square, rectangular, triangular, irregularly shaped, polygonal or any other desired shape. The mesh support comprises a material selected from copper, gold, nickel, rhodium, molybdenum, silicon, carbon, metal and metal alloys (e.g., stainless steel) etc. The mesh support maybe surface modified to achieve desired characteristics. In some embodiments the mesh support comprise an electron transparent material. As used herein, the term “electron transparent” refers to a material that allows for pass-through of electrons with minimal scattering or absorption. Exemplary electron transparent materials include materials carbon, gold, and graphene oxide.

The wires/bands of the mesh support may have cross-sectional dimensions of 1-200 μm (e.g., 1 μm, 2 μm, 5 μm, 10 μm, 20 μm, 50 μm, 100 μm, 200 μm or ranges therebetween) and may have gaps between adjacent wires/bands of 25-400 μm (e.g., 25 μm, 50 μm, 75 μm, 100 μm, 150 μm, 200 μm, 250 μm, 400 μm or ranges therebetween). In some embodiments, the gaps between the wires/bands create mesh spaces. In some embodiments, the mesh support comprises an outer rim where the rim width is 50-800 μm (e.g., 50 μm, 75 μm, 100 μm, 150 μm, 200 μm, 250 μm, 800 μm and ranges therebetween).

Provided herein are lipid bilayers extending across apertures through substrates. In some embodiments the entire aperture is occluded by the lipid bilayer extending thereacross. In some embodiments, a single thickness of bilayer extends across the aperture. In some embodiments, the bilayer terminates at the periphery of the aperture. In some embodiments, the bilayer continues from the aperture to coat the surface of the substrate (e.g., top and/or bottom of the substrate). In some embodiments, the bilayer splits at the periphery of the aperture, with a lipid monolayer extending above and/or below the substrate. In some embodiments, the thickness of the bilayer is determined by the lipid composition (and optionally other components) of the bilayer. In some embodiments, a substrate and bilayer composition are selected to achieve a bilayer extending across the apertures of a substrate in a desired conformation. In some embodiments, the thickness of the substrate (e.g., at the periphery of the aperture, across the entire substrate, etc.) is approximately equal (e.g., plus/minus 20%, 15%, 10%, 5%, 1% or ranges therebetween) to the thickness of the lipid bilayer. In some embodiments, the thickness of the substrate (e.g., at the periphery of the aperture, across the entire substrate, etc.) is greater than equal the thickness of the lipid bilayer by 1-40 nm (e.g., 1 nm, 2 nm, 5 nm, 10 nm, 15 nm, 20 nm, 30 nm, 40 nm, or ranges therebetween). In some embodiments, the thickness of the substrate (e.g., at the periphery of the aperture, across the entire substrate, etc.) is less than the thickness of the lipid bilayer by about 1-8 nm (e.g., 1 nm, 2 nm, 3 nm, 4 nm, 5 nm, 6 nm, 7 nm, 8 nm, or ranges therebetween).). In some embodiments, the thickness of the substrate (e.g., at the periphery of the aperture, across the entire substrate, etc.) is achieved to avoid hydrophobic mismatch between the hydrophobic substrate and hydrophobic core (formed by lipid chains, sterols etc.) of the lipid bilayer.

In some embodiments, the lipid bilayers herein (e.g., assembled and/or extending across apertures) are primarily composed of lipids, including phospholipids, modified lipids, sterols, and other optional components that contribute to their structural integrity, fluidity, and functionality. Lipids are the primary structural components of lipid bilayers, and form the foundational structure of the membrane and contribute to its physicochemical properties, such as fluidity, permeability, and stability. In some embodiments, the lipid bilayers herein comprise one or more phospholipids. Phospholipids are the most common type of lipid in bilayers and are characterized by their amphiphilic nature, with hydrophilic heads and hydrophobic tails. Exemplary phospholipids that find use in the bilayers herein include, but are not limited to phosphatidylcholine (PC), phosphatidylethanolamine (PE), phosphatidylserine (PS), phosphatidylinositol (PI), and sphingomyelin (SM). In some embodiments, the lipid bilayers herein comprise one or more glycolipids, or lipids with sugar moieties, such as gangliosides and cerebrosides. In some embodiments, the lipid bilayers herein comprise one or more sterols, which can affect fluidity, curvature, and stability of a bilayer. Exemplary sterols that may find use in bilayers herein include, but are not limited to cholesterol, sitosterol, lanosterol, ergosterol.

Lipid bilayers herein may also comprise one or more of sphingolipids (e.g., sphingosine, ceramide, etc.), modified lipids (e.g., amino-phospholipids (e.g., 1,2-Diolcoyl-sn-glycero-3-phosphoethanolamine (DOPE), etc.), PEGylated lipids (e.g., 1,2-Diolcoyl-sn-glycero-3-phosphoethanolamine-N-[methoxy (polyethylene glycol)-2000] (DOPE-PEG), etc.), fluorescently-tagged lipids (e.g., N-(7-Nitro-2-1,3-benzoxadiazol-4-yl) phosphatidylethanolamine (NBD-PE), etc.), diacylglycerols, lysophospholipids, etc. Lipid bilayers herein may also comprise a natural cell membrane lipid extract or a complete/partial lipidome. Other components of a lipid bilayer herein may include integral membrane proteins, peripheral membrane proteins, peptides, buffers, salts, metal ions, etc. The full scope of embodiments herein is not limited by the variety of bilayer components. In some embodiments particularly for the study or development of various embodiments herein, fluorescently-labeled or otherwise detectably labeled lipids or other components of lipid bilayers may be included.

In some embodiments, a lipid bilayer herein is between 2 nm and 12 nanometers thick, depending largely upon the components used to assemble the lipid bilayer. In general, phospholipid bilayers are 4-5 nm thick, sphingolipid bilayers are 4-6 nm thick, cholesterol-containing bilayers are 4-6 nm thick, but lipid bilayers with modified lipids, such as those with larger/smaller headgroups or longer/smaller acyl chains, may have thicknesses outside or less than the typical range. For instance, bilayers with very long acyl chains may exceed 6 nm. Typically, bilayer thickness is largely determined by the length of the lipid's hydrophobic fatty acid chains and the overall lipid composition.

In some embodiments, formation of a lipid bilayer within an aperture, according to certain methods herein, requires a detergent. Detergents, also known as surfactants, are amphipathic molecules that contain both polar (hydrophilic) and non-polar (hydrophobic) regions, allowing them to solubilize hydrophobic molecules in aqueous solution (e.g., water). Suitable detergents that may find use in assembly of lipid bilayers according to the embodiments herein include, for example, pluronic F-127, Tween 20, Tween 80, Triton X-100, sodium dodecyl sulfate (SDS), cetyl trimethyl-ammonium bromide (CTAB), digitonin, n-octyl-β-D-glucoside etc. In some embodiments, various surfactants may be employed in the methods herein (e.g., as detergents and/or with other detergents). Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine. In some embodiments, one or more emulsifiers are used in assembling lipid bilayers according to the methods herein. Suitable emulsifiers include, for example, lecithin, saponins, ammonium phosphatide (E442), polyglycerol polyricinoleate (E476), diacetyl tartaric acid ester of monoglyceride, DOWSIL ES-5300 Formulation Aid (a non-diluted and low viscosity silicone surfactant designed to produce stable water-in-silicone emulsions and water-in-oil emulsions), etc.

To assemble a lipid bilayer in a grid aperture (e.g., of a holey carbon grid or holey grid of any material, for example, but not limited to, chemically modified gold, Teflon or any chemically modifiable surface like SiO₂), similar principles and methods are employed which are used for assembling lipid bilayers in nanodiscs or solid apertures. In some embodiments, methods involve combining lipids and any other bilayer components described herein or understood in the field (collectively referred to herein as the “lipid bilayer components”) and detergent(s) (and/or surfactants and/or emulsifiers), collectively referred to herein as the “assembly reagents” in the presence of the holey grid, and then removing the detergent and/or other assembly reagents and allowing a bilayer of the lipid components to form across the aperture of the holey grid. Upon removal of the detergent and/or other assembly reagents at a suitable rate (e.g., over the course of 5 minutes, 10 minutes, 15 minutes, 20 minutes, 30 minutes, 1 hour, 2 hours, 4 hours, 8 hours, 12 hours, 24 hours, 48 hours, 72 hours 96 hours, or more or ranges therebetween), the bilayer forms across the apertures, as described herein. In some embodiments, the bilayer is limited to the aperture space. In other embodiments, the bilayer (or monolayer(s) thereof) extends onto the substrate within which the aperture is formed.

Removal of the detergent (and/or other assembly reagents (e.g., surfactant(s), emulsifier(s), ions, etc.) from the lipid containing solution/mixture in the presence of the aperture-containing substrate material results in the formation of the bilayer across the aperture(s). The detergent (and/or other assembly reagents) can be removed from the solution/mixture by any suitable method, including but not limited to those described herein.

One method of removing assembly component(s) (e.g., detergent) from the lipid bilayer components is by passive diffusion across a permeable surface. In some embodiments, the lipid bilayer components and assembly component(s) (e.g., detergent) are placed within a vessel capable of also containing one or more holey grids. In some embodiments, the vessel comprises one or more walls or surfaces that are permeable to molecules, including one or more of the assembly components (e.g., the detergent(s), etc.). In some embodiments, the lipid bilayer components, assembly component(s) (e.g., detergent), and holey grid(s) are placed within the vessel having one or more permeable surfaces (e.g., a dialysis membrane); the vessel is placed in or contacted with a solution/mixture with a lower concentration or absence of one or more of the assembly components (e.g., the detergent(s), etc.); and the assembly components (e.g., the detergent(s), etc.) are allowed to diffuse from inside the vessel across the permeable surface, thereby reducing the concentration of the assembly component(s) (e.g., the detergent(s), etc.) in the vessel and in contact with the lipid bilayer components and aperture(s) of the holey grid. In some embodiments, the solution/mixture outside of the vessel is changed one or more times (or circulated or otherwise refreshed) to result in reduction of the concentration of assembly component(s) (e.g., the detergent(s), etc.) in the vessel to near 0 mM (e.g., <10 mM, <1 mM, <100 μM, <10 μM, <1 μM, <1 nM, <1pM or less). In some embodiments the volume of mixture/solution outside the vessel is much larger (e.g., 10×, 20×, 50×, 100×, 200×, 500×, 1000×, or more) than the volume inside the vessel.

A second method of removing assembly component(s) (e.g., detergent) from the lipid bilayer components is by capture and/or sequestration of the assembly components (e.g., detergent). In some embodiments, the lipid bilayer components and assembly component(s) (e.g., detergent) are combined with one or more holey grids. In some embodiments, an adsorbing agent is added. The adsorbing agent is a molecular entity or material capable of binding up the desired assembly component(s) (e.g., detergent). Exemplary adsorbing agents include, but are not limited to polystyrene, polyethylene glycol, polysorbate, polyvinyl alcohol, poly(ethyleneimine), sepharose, silica resins, ion exchange resins, activated charcoal, fullerene derivatives, cyclodextrins, silica gel, alumina, and bovine serum albumin. The adsorbing agent sequesters the assembly component(s) (e.g., detergent) away from the holey grid, creating a local reduction in the concentration of assembly component(s) at the apertures of the holey grid and allowing the bilayer to form across the apertures as the local concentration of assembly component(s) (e.g., detergent) is reduced (e.g., to near 0 mM (e.g., <10 mM, <1 mM, <100 μM, <10 μM, <1μM, <1 nM, <1pM or less).

In some embodiments, the holes in the holey grid are painted with an organic solvent containing lipid bilayer components. Lipids dissolved in an organic solvent (e.g., decane, squalene) are applied on the grid and after the solvent is dried the grid is placed in a buffer solution and more of the solvent containing lipids are applied on the grid until a lipid bilayer is formed. In another method, a folding technique, a lipid monolayer at the air-water interface gets folded, forming a bilayer, when the aperture/hole is raised above the water surface.

In some embodiments, the lipid bilayers within apertures of holey grids provide an environment for the analysis of membrane-bound,-embedded,-linked, and-associated proteins, complexes, macromolecules, etc. In some embodiments, provided herein are holey grids with lipid bilayers extending across the apertures thereof, and further comprising a macromolecule-of-interest bound to, linked to, embedded within, and/or associated with the lipid bilayer. In some embodiments, the presence of the macromolecule-of-interest on or within the lipid bilayer of the holey grid allows for structural (and/or functional) analysis of the macromolecule-of-interest within an environment that mimics the native state of the macromolecule-of-interest. In some embodiments, a macromolecule-of-interest is a protein. In some embodiments, the protein is a transmembrane protein (e.g., single-pass transmembrane protein or multi-pass transmembrane protein), integral membrane protein, peripheral membrane protein, lipid-anchored protein, membrane-associated enzyme, membrane transporter, receptor protein, cell adhesion molecule, membrane-bound receptor, structural protein, soluble protein etc. In some embodiments, a macromolecule-of-interest is a glycans (glycoconjugates), glycolipid, membrane-associate lipid complex, vesicle-coated complex, membrane-bound RNA complex, ribonucleoprotein, protein complex with a membrane protein, proteoliposome attached to the lipid bilayer in aperture etc. Embodiments herein are not limited by the identity of the macromolecule-of-interest associated with the lipid bilayer of the holey grids described herein.

In some embodiments, provided herein are methods of introducing a macromolecule-of-interest into/onto the lipid bilayers described herein. In some embodiments, one or more macromolecule(s)-of-interest are included with the lipid-bilayer components and assembly components during assembly of the lipid bilayer within the apertures. Without being bound by any particular mechanism of assembly, it is contemplated that as the lipid bilayer forms, the macromolecule(s)-of-interest associate with the lipid bilayer according to their natural proclivities to do so (e.g., to span the bilayer, associate with the surface of the bilayer, to be anchored into the bilayer, to integrate into the bilayer, etc.). In other embodiments, macromolecule(s)-of-interest are added to a holey grid after assembly of the lipid bilayer within the apertures thereof. Various techniques are available for addition of a macromolecule-of-interest to an established lipid bilayer. For example, in some embodiments, a macromolecule-of-interest is solubilized (e.g., in a mild detergent) and then directly added to the established lipid bilayers within the apertures. In other embodiments, the macromolecule-of-interest is added to the lipid bilayer in a carrier that facilitates transfer of the macromolecule-of-interest to the lipid bilayer within the aperture. Suitable carriers include liposomes, vesicles (e.g., unilamellar vesicles, multilamellar vesicles, etc.), proteoliposomes, etc. In some embodiments, a macromolecule-of-interest is electroporated, sonicated, extruded, etc. into the lipid bilayer. In some embodiments, a macromolecule-of-interest is embedded, linked or fused to a lipid bilayer component (e.g., a lipid anchor) for delivery onto/into the lipid bilayer. In some embodiments, a macromolecule-of-interest is first incorporated into a nanodisc and then transferred therefrom into the lipid bilayer of a holey grid. In some embodiments, the macromolecule-of-interest detects a receptor entity on the lipids or any assembled lipid bilayer component for binding. The full scope of embodiments herein are not limited by the techniques used to incorporate macromolecule(s)-of-interest into the bilayers of the holey grids herein.

Using any of the methods of described elsewhere herein, in some embodiments, the macromolecule-of-interest can embed or bind to the lipid monolayer or lipid bilayer formed on the substrate (apart from the apertures) and studied for structural and/or functional analysis.

Nanodiscs utilized in cryoEM for membrane proteins offer a native lipid bilayer environment. However, sample heterogeneity on grids often impacts SPA. Moreover, the use of nanodiscs entails additional complexities such as the expression and purification of scaffold proteins, size limitations for nanodiscs vs. proteins, and the potential incorporation of more than one protein per nanodisc. Alternatively, proteoliposomes can be directly employed for SPA in cryoEM. However, their size may influence ice thickness, and certain protein structures are sensitive to curvature. However, proteoliposomes and/or nanodiscs (as well as other carriers) can also be used to associate/embed proteins (or other macromolecules) in the lipid bilayers assembled in the apertures of holey grids herein.

In some embodiments, methods herein comprise one or more wash steps. Wash steps are employed to remove contaminants, reagents, unincorporated components, etc. from mixtures/solutions described herein. A wash step may be conducted before and/or after any of the method steps described herein. For example, holey grid may be washed after assembly of the lipid bilayer within the apertures therein to remove excess/unincorporated assembly components, lipid bilayer components, free liposomes or proteoliposomes, ligands, drugs, and/or macromolecules of interest. A wash step may be employed to change the solution/mixture (e.g., buffer, salt concentration, etc.) that a component described herein (e.g., holey grid) is contained within.

The holey grids described herein (e.g., with lipid bilayers and embedded/bind macromolecules of interest in the apertures thereof) are not limited by the analytical/biophysical techniques used to analyze the samples therein. For example, holey grids herein may find use with transmission electron microscopy (TEM), Cryo-electron microscopy (Cryo-EM), electron diffraction (ED), energy-dispersive X-ray spectroscopy (EDX or EDS), scanning electron microscopy (SEM), atomic force microscopy (AFM), Raman spectroscopy, X-ray photoelectron spectroscopy (XPS), fluorescence microscopy, total internal reflection fluorescence microscopy (TIRF), small-angle X-ray scattering (SAXS), surface plasmon resonance (SPR), circular dichroism (CD) spectroscopy, nuclear magnetic resonance (NMR) spectroscopy, mass spectrometry (MS), fluorescence correlation spectroscopy (FCS), electron tomography (ET), etc. Methods of conducting such techniques using the holey grids described herein (e.g., with a macromolecule-of-interest on/in a lipid bilayer assembled in the apertures thereof) are provided herein.

In some embodiments, provided herein are kits comprising the holey grids described herein (e.g., with a lipid bilayer assembled in the apertures thereof). Such kits may further comprise instructions for use, reagents for addition of a macromolecule-of-interest, etc. In some embodiments, provided herein are kits comprising holey grids and components (e.g., lipid components, assembly components, macromolecule addition components, etc.) for preparing a lipid bilayer and/or introducing a macromolecule-of-interest to the apertures of the holey grid.

### Experimental

Experiments described herein employ an exemplary carbon surface as a model material but the methodologies described here are applicable to any kind of material.

### Example 1

### Assembling Free-Standing Lipid Bilayer in Holey Grids

Free-standing lipid bilayers are assembled in the apertures of holey grids (e.g., using commercially available Quantifoil or C-Flat grids) or in the apertures of other suitable substrates (any thickness; nanometer to micrometer), for example but not limited to gold or Teflon any other material or any chemically modifiable surface like SiO₂ and supported by a mesh, which can be of any thickness; nanometer to millimeter, made of any material, for example, but not limited to, copper, gold, nickel, any metal, any alloy etc.) suitable for cryoEM. Furthermore, the hole can be of any size (nanometer to micrometer scales) and shape (circular, oval, square, polygonal etc.) and the mesh support can be of any size (spacing/inch) and shape (circular, oval, square, polygonal etc.).

Similar protocols which are typically used for preparing nanodiscs are employed here to assemble the lipid bilayers in holey grids. Lipid assembly is confirmed using transmission electron microscopes (e.g, cryo-electron microscopes Glacios and Krios or any).

Experiments are conducted during development of embodiments herein to establish methods to assemble free-standing lipid bilayer inside the holes of holey carbon grids. Different types of commercially available grids (Quantifoil and C-flat) are used for initial tests. These commercial grids typically feature a carbon thickness ranging from 10 to 40 nm, with hole diameters spanning from 0.6 to 17 μm, supported on copper or gold mesh (200-400 squares/inch). Commonly known techniques to assemble lipids will be used to assemble lipid bilayers in the holes of holey carbon grids and are described below. But these methods can be applied to any suitable grids, for example but not limited to gold or Teflon any other material or any chemically modifiable surface like SiO₂ (where these surfaces can be of any thickness; nanometer to micrometer) and supported by a mesh made, which can be of any thickness; nanometer to millimeter, of any material, for example, but not limited to, copper, gold, nickel, any metal, any alloy etc.

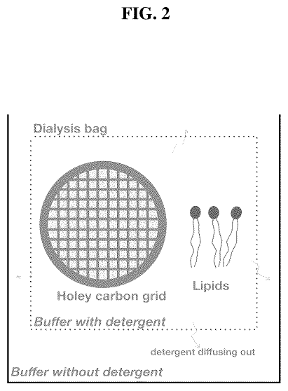

Similar to the methods described for the formation of nanodiscs, the holey carbon grids (not glow discharged to keep them hydrophobic) are placed in a solution containing a mixture of lipids, including a small percentage of fluorophore lipids, and detergents (various lipid types and their compositions and detergent types and their compositions are evaluated). To form the lipid bilayer, detergents are removed using two different approaches. In the first approach, the whole mix (detergents, lipids, grids) are placed inside a dialysis bag immersed in a buffer solution without detergent (at room temperature or 4° C. or to be optimized) to allow the detergent diffuse out from the dialysis bag (FIG. 2). In the second approach biobeads are added to the whole mix; biobeads are known to adsorb the detergents.

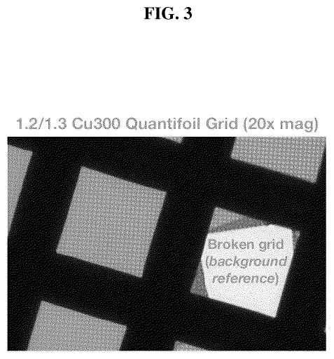

Using either approach, after removing the detergent, the formation of lipid bilayer inside the holes of holey grids is evaluated and observed by fluorescence, at different time stamps over 48 h, by placing the grids under an inverted light microscope (or any suitable microscope can be used) with an excitation/emission channel corresponding to the fluorophore lipids; with inverted microscope accidentally hitting the grids by the objective lens is not possible and the objectives are below a transparent slide holding the grid. Light microscope can be used to observe the holes (see FIG. 3) and to observe fluorescence inside the holes if a bilayer is formed which will be finally evaluated in cryoEM after plunge freezing the grids (for example, by manual method or using a Vitrobot or Lieca GP2 for plunge freezing, cryoEM Glacios for screening and cryoEM Krios for data collection). For plunge freezing different blotting conditions (blot force and blot time) are tested using a Vitrobot and also one sided blotting is performed manually by placing a blotting paper on the metal side of the grid or at the edge of the grid to avoid potential damage to the lipid bilayer membrane on the carbon side or by using Leica GP2 instrument.

This approach will help the direct assembly of a free-standing lipid bilayer with many copies of membrane protein or membrane embedded/attached particles in each hole in a single step which, after plunge freezing, can be used directly for cryoEM.

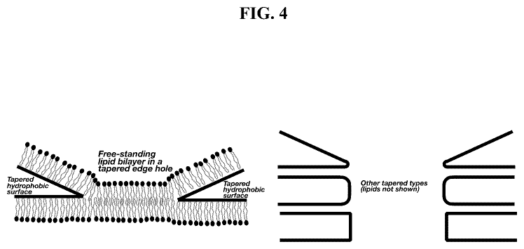

In some customized embodiments, stable free floating lipid bilayers, using a similar approach described above, are assembled on tapered edges of the carbon layer of any thickness; nanometer to micrometer (or any other material, for example, but not limited to gold or Teflon any other material or any chemically modifiable surface like SiO₂ and supported by a mesh, which can be of any thickness; nanometer to millimeter, made of any material, for example, but not limited to, copper, gold, nickel, any metal, any alloy etc) with variable thickness which can be in nanometer to micrometer range. The tapered edge helps in reducing the septum-annulus contact angle which overall stabilizes the lipid bilayer and the extent of tapering (angle, curved, etc.) can be varied (FIG. 4). Similarly, painting (applying a solvent with lipids) and folding method (by raising the grid through an air-water interface containing a lipid monolayer) can be applied for any type of grids with flat or tapered surface.

### Computational Modelling and Molecular Dynamics Simulations

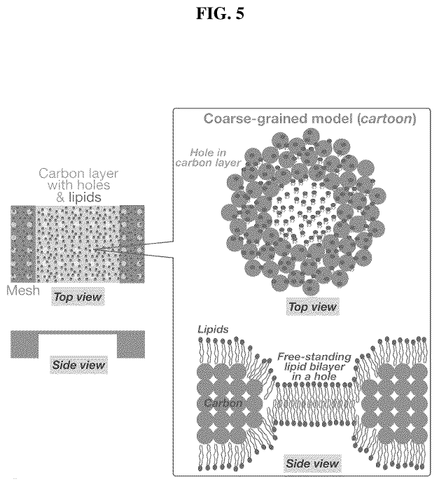

MARTINI based coarse-grained computational molecular dynamics (MD) simulations are conducted of the holey carbon hole with lipids (FIG. 5) but similar models, simulations and understandings so gained can be used for any type of holey grids (any layer material of any thickness and dimension; nanometer to micrometer, for example, but not limited to carbon, gold or Teflon any other material or any chemically modifiable surface like SiO₂ and supported by a mesh made of any material (which can be of any thickness and dimension; nanometer to millimeter), for example, but not limited to, copper, gold, nickel, any metal, any alloy etc).

MARTINI based models have been widely used for lipid bilayers and other biological systems. Experiments test the stability of lipid bilayer using the carbon hole models of different diameters and thickness. 50 nm-1 μm range diameter holes with different carbon thickness (4 to 20 nm) are simulated to study lipid bilayer assembly and to understand the interaction, stability and lipid dynamics at the septum annulus interface. Lipid bilayer properties (bilayer thickness, lipid order parameters etc.) are calculated. Furthermore to support the experiments, mixture of different lipid chain lengths, chemical types, functional groups etc. are tested.

### Quantitative and Qualitative Performance Measures

Holey grids showing fluorescence (using an inverted light microscope or any suitable microscope or fluorescence imaging instrument) in the holes are plunge frozen (with and without blotting) in ethane or a mixture of ethane and propane and the grids are screened to observe the qualitative features such as lipid bilayer formation, ice thickness using cryoEM (e.g. Glacios). Images and tomographs (at different angles) are collected using the cryoEM (e.g. Krios) to measure the bilayer thickness. For cryoEM data analyses and tomogram reconstruction EMAN2 and IMOD software is used (or any other suitable software can be used). For MD simulations the simulated lipid bilayer properties in the holes are compared with the simulated free and bulk lipid bilayers (not supported by a scaffold or a surface) and with the lipid bilayer properties (e.g. thickness) observed in the holey grids using cryoEM. It has been reported that large nanodiscs, ˜45 nm, support lipid bilayers with their properties similar to bulk lipid bilayers. The bilayer thickness calculated from computational modelling and simulations is compared with the cryoEM data, which further validates the models. MD simulations are done using GROMACS MD software or any other suitable MD software.

### Example 2

### Assembling Free-Standing Lipid Bilayer in Holey Carbon Grids With Membrane Proteins

Using optimized conditions determined from Example 1, free-standing lipid bilayers with the membrane proteins are assembled in the holes of holey grids using commercially available grids or customised. These holey grids can have flat or tapered holes formed with carbon layer or any other material (which can be of any thickness; nanometer to micrometer), for example, but not limited to gold or Teflon any other material or any chemically modifiable surface like SiO₂ and supported by a mesh, which can be of any thickness; nanometer to millimeter, made of any material, for example, but not limited to, copper, gold, nickel, any metal, any alloy etc. Furthermore, the hole can be of any size (nanometer to micrometer scales) and shape (circular, oval, square, polygonal etc.) and the mesh support can be of any size (spacing/inch) and shape (circular, oval, square, polygonal etc.).

Similar protocols to those established in Example 1 are followed, with the inclusion of membrane proteins during the assembly process. Confirmation of lipid assembly featuring a uniform distribution of protein particles is conducted using cryoEM Glacios and Krios. CryoEM analyses is carried out on the data collected using Krios (for example, with K3 detectors, energy filters).

Experiments establish a complete protocol from assembling a free-standing lipid bilayers to cryoEM analyses to help resolving the membrane protein structure in a native lipid environment which maybe applied for any membrane protein as a generic method.

Similar experiments and approaches can be applied for any membrane attached or embedded particles which can be any type of synthetic or natural molecules, for example, but not limited to, hydrophobic DNA and DNA nanostructures, peptides, polymers etc.).

### Membrane Protein Insertion

Using the optimized conditions obtained in Example 1 to assemble a free-standing lipid bilayer, the holey grids are placed inside a dialysis bag containing purified membrane protein of interest (or can be any type of synthetic or natural molecules, for example, but not limited to, hydrophobic DNA and DNA nanostructures, peptides, polymers etc), with a mixture of lipids, including a small percentage of fluorophore lipids, and detergents (different lipid compositions and detergents are tested). After dialysing out the detergent the formation of lipid bilayer is evaluated, at different time stamps, e.g., over 48 h, using a fluorescence light microscope with a channel corresponding to the fluorophore lipids. Experiments are conducted to remove detergents either using a dialysis bag method or biobeads in the presence of membrane proteins and lipids. The grids are plunged frozen in ethane (or a mixture of ethane and propane) using Vitrobot (without or with blotting; different blot force and time conditions or using Leica GP2 instrument or manually) and screened with cryoEM (e.g., Glacios). Screened grids showing thin ice with uniform distribution of particles (e.g., membrane proteins) in the holes are further used for data collection with cryoEM (e.g. Krios with K3 detectors and energy filters).

For the grids prepared with alternative strategies proteoliposomes are incubated with the lipid bilayer membrane assembled either with painting or folding method. Proteoliposomes are reconstituted by mixing membrane proteins with lipids and detergents and later detergent can be removed by dialysis. Fusion of proteoliposome with the lipid bilayer depends on many factors including the surface properties and size of the aperture, the size of the liposome and the lipid composition (different lipid compositions will be explored). Instead of membrane proteins, other molecules, any type of synthetic or natural molecules, for example, but not limited to, hydrophobic DNA and DNA nanostructures, peptides, polymers etc, can also be used to form the liposomes for their insertion in the lipid bilayer formed by alternative strategies.

Membrane proteins that are available commercially in purified forms and whose structures have been resolved using cryoEM approaches are tested. For instance, outer membrane protein F; ompF (˜110 kDa trimer), stabilized in nanodiscs, has been resolved at 2.54 Å and acriflavine resistance Protein B; acrB (˜350 kDa trimer), stabilized as proteoliposome, has been solved at 3.9 Å. Both these proteins are available commercially and these proteins can be purified in the lab. Nonetheless, any membrane protein or any type of synthetic or natural molecules, for example, but not limited to, hydrophobic DNA and DNA nanostructures, peptides, polymers etc., can be used.

### CryoEM Analyses

Experiments are conducted during development of embodiments herein to assemble a planar lipid bilayer with membrane proteins (or any type of synthetic or natural molecules, for example, but not limited to, hydrophobic DNA and DNA nanostructures, peptides, polymers etc). In some embodiments, membrane embedded/attached molecules have a preferred orientation but with random in-plane rotation while facing up or down randomly (across the bilayer). Using a conventional approach for data collection for single particle analyses, where grids remain perpendicular to the electron beam, it may not be able to sample a complete Fourier space. Recently, a workflow has been reported to collect data at different angles and employ image processing techniques resulting in a 3 Å resolution map for a protein exhibiting preferred orientation. This method is applicable to integral membrane proteins or membrane embedded/attached molecules as well. In some embodiments, methods here in adopt a similar protocol for collecting images at different tilt angles to achieve improved Fourier space sampling, which is required for isotropic resolution and perform data analyses using cryoSPARC and RELION software or any other suitable software. Moreover, membrane proteins or membrane embedded/attached molecules in the fluidic lipid bilayer are not rigidly oriented and thus are expected to present slightly different orientations in terms of their tilt angles with respect to the lipid bilayer. MD simulations of the membrane proteins indicate that transmembrane α-helical peptides typically exhibit a Gaussian distribution of tilt angles along the preferred orientations, whereas bacterial outer membrane proteins, barrels formed by β-strands, display tilt angle dynamics. Also, larger membrane proteins tend to have bending motions as shown with MD simulations. Additionally, MD simulations of membrane embedded DNA nanopores also show large variation in tilt angles. These dynamic motions will be captured during the plunge freezing. Variation in the tilt angles of the membrane proteins or membrane embedded/attached molecules relative to the lipid bilayer surface, coupled with stage tilting of the grids will facilitate obtaining orientation distributions of the imaged particles, thereby enhancing Fourier sampling. Moreover, images are acquired at different angles to collect tilt-series at suitable electron doses to construct the tomograms. Utilizing subtomogram averaging (STA) method, employing EMAN2 software and others, <3-5 Å resolution is achievable. Depending on the factors such as number of particles, purity, molecule size, orientations and the quality of tomogram reconstruction, resolutions as high as 2.8 Å are feasible with STA.

### Quantitative and Qualitative Performance Measures

The holey grids showing fluorescence (observed with a suitable light microscope or imaging instrument) in the holes are plunge frozen (with and without blotting) in ethane or mixture of ethane with propane, and the grids are screened to observe the qualitative features such as lipid bilayer formation, vitreous ice, uniform distribution of particles using cryoEM (e.g., Glacios). Next, the data (without or with tilt angle imaging) is collected using cryoEM (e.g. Krios with K3 detectors, energy filters) and the final quantitative performance is evaluated from the resolution of cryoEM maps (Fourier shell correlation); minimal cone angle and isotropic resolution. Data analyses will be performed using EMAN2, IMOD, RELION, cryoSPARC or any other suitable software.

To avoid a potential issue of having a crowded population (affecting SPA and STA) of membrane proteins or membrane embedded/attached molecules in the free-standing lipid bilayers, the bilayer assembly is tested using different concentrations of molecules during the assembly process. Any non-uniform distribution of particles in the lipid bilayers is resolved using different lipid mixtures (if islands of proteins are observed) and different blotting conditions (if more particles at the edges are observed).

In cryoEM, tilting the grids may induce doming effects, cause beam-induced drifting of the particles, and increase ice-thickness at higher tilt angles. These issues are tested and balanced and at the same time to ensure full coverage of Fourier space. Resolution maps collected at various tilt angles are compared with those from untilted samples. For highly oriented samples, a cone effect may be observed, leading to anisotropic resolution maps. As described above, membrane proteins or membrane embedded/attached particles are not typically rigidly oriented, and their tilt angles with respect to the lipid bilayer can vary. Nonetheless, strategies herein may be useful for resolving extracellular domains and their complex with a ligand or drug molecule of interest.

### REFERENCES

The following reference are herein incorporated by reference in their entireties.
(1) Overington, J. P.; Al-Lazikani, B.; Hopkins, A. L. How Many Drug Targets Are There? Nat. Rev. Drug Discov. 2006, 5, 993-996.
(2) Henderson, R.; Baldwin, J. M.; Ceska, T. A.; Zemlin, F.; Beckmann, E.; Downing, K. H. Model for the Structure of Bacteriorhodopsin Based on High-Resolution Electron Cryo-Microscopy. J. Mol. Biol. 1990, 213, 899-929.
(3) Brzezinski, P. Scientifc Background on the Nobel Prize in Chemistry 2017 THE DEVELOPMENT OF CRYO-ELECTRON MICROSCOPY https://www.nobelprize.org/uploads/2018/06/advanced-chemistryprize2017.pdf (accessed Feb. 26, 2017).
(4) Callaway, E. Revolutionary Cryo-EM Is Taking over Structural Biology. Nature 2020, 578, 201.
(5) Liao, M.; Cao, E.; Julius, D.; Cheng, Y. Structure of the TRPV1 Ion Channel Determined by Electron Cryo-Microscopy. Nature 2013, 504, 107-112.
(6) Efremov, R. G.; Gatsogiannis, C.; Raunser, S. Chapter One-Lipid Nanodiscs as a Tool for High-Resolution Structure Determination of Membrane Proteins by Single-Particle Cryo-EM. In A Structure-Function Toolbox for Membrane Transporter and Channels; Ziegler, C. B. T.-M. in E., Ed.; Academic Press, 2017; Vol. 594, pp 1-30.
(7) Denisov, I. G.; Grinkova, Y. V; Lazarides, A. A.; Sligar, S. G. Directed Self-Assembly of Monodisperse Phospholipid Bilayer Nanodiscs with Controlled Size. J. Am. Chem. Soc. 2004, 126, 3477-3487.
(8) Gatsogiannis, C.; Lang, A. E.; Meusch, D.; Pfaumann, V.; Hofnagel, O.; Benz, R.; Aktories, K.; Raunser, S. A Syringe-like Injection Mechanism in Photorhabdus Luminescens Toxins. Nature 2013, 495, 520-523.
(9) Shi, D.; Nannenga, B. L.; Iadanza, M. G.; Gonen, T. Three-Dimensional Electron Crystallography of Protein Microcrystals. Elife 2013, 2, e01345.
(10) Martynowycz, M. W.; Khan, F.; Hattne, J.; Abramson, J.; Gonen, T. MicroED Structure of Lipid-Embedded Mammalian Mitochondrial Voltage-Dependent Anion Channel. Proc. Natl. Acad. Sci. 2020, 117, 32380-32385.
(11) Martynowycz, M. W.; Shiriaeva, A.; Ge, X.; Hattne, J.; Nannenga, B. L.; Cherezov, V.; Gonen, T. MicroED Structure of the Human Adenosine Receptor Determined from a Single Nanocrystal in LCP. Proc. Natl. Acad. Sci. 2021, 118, e2106041118.
(12) Mio, K.; Sato, C. Lipid Environment of Membrane Proteins in Cryo-EM Based Structural Analysis. Biophys. Rev. 2018, 10, 307-316.
(13) Nasr, M. L.; Baptista, D.; Strauss, M.; Sun, Z.-Y. J.; Grigoriu, S.; Huser, S.; Plückthun, A.; Hagn, F.; Walz, T.; Hogle, J. M.; Wagner, G. Covalently Circularized Nanodiscs for Studying Membrane Proteins and Viral Entry. Nat. Methods 2017, 14, 49-52.
(14) Kalsi, S.; Powl, A. M.; Wallace, B. A.; Morgan, H.; de Planque, M. R. R. Shaped Apertures in Photoresist Films Enhance the Lifetime and Mechanical Stability of Suspended Lipid Bilayers. Biophys. J. 2014, 106, 1650-1659.
(15) Hirano-Iwata, A.; Aoto, K.; Oshima, A.; Taira, T.; Yamaguchi, R.; Kimura, Y.; Niwano, M. Free-Standing Lipid Bilayers in Silicon Chips-Membrane Stabilization Based on Microfabricated Apertures with a Nanometer-Scale Smoothness. Langmuir 2010, 26, 1949-1952.
(16) Souza, P. C. T.; Alessandri, R.; Barnoud, J.; Thallmair, S.; Faustino, I.; Grünewald, F.; Patmanidis, I.; Abdizadeh, H.; Bruininks, B. M. H.; Wassenaar, T. A.; Kroon, P. C.; Melcr, J.; Nieto, V.; Corradi, V.; Khan, H. M.; Domański, J.; Javanainen, M.; Martinez-Seara, H.; Reuter, N.; Best, R. B.; Vattulainen, I.; Monticelli, L.; Periole, X.; Tieleman, D. P.; de Vries, A. H.; Marrink, S. J. Martini 3: A General Purpose Force Field for Coarse-Grained Molecular Dynamics. Nat. Methods 2021, 18, 382-388.
(17) Tang, G.; Peng, L.; Baldwin, P. R.; Mann, D. S.; Jiang, W.; Rees, I.; Ludtke, S. J. EMAN2: An Extensible Image Processing Suite for Electron Microscopy. J. Struct. Biol. 2007, 157, 38-46.
(18) Kremer, J. R.; Mastronarde, D. N.; McIntosh, J. R. Computer Visualization of Three Dimensional Image Data Using IMOD. J. Struct. Biol. 1996, 116, 71-76.
(19) Maingi, V.; Rothemund, P. W. K. Properties of DNA- and Protein-Scaffolded Lipid Nanodiscs. ACS Nano 2021, 15, 751-764.
(20) Berendsen, H. J. C.; van der Spoel, D.; van Drunen, R. GROMACS: A Message-Passing Parallel Molecular Dynamics Implementation. Comput. Phys. Commun. 1995, 91, 43-56.
(21) Kongsuphol, P.; Fang, K. B.; Ding, Z. Lipid Bilayer Technologies in Ion Channel Recordings and Their Potential in Drug Screening Assay. Sensors Actuators B Chem. 2013, 185, 530-542.
(22) Bartsch, P.; Walter, C.; Selenschik, P.; Honigmann, A.; Wagner, R. Horizontal Bilayer for Electrical and Optical Recordings. Materials. 2012, U.S. Plant Pat. No. 2,705-2730.
(23) Marr, C. R.; Benlekbir, S.; Rubinstein, J. L. Fabrication of Carbon Films with ˜500 nm Holes for Cryo-EM with a Direct Detector Device. J. Struct. Biol. 2014, 185, 42-47.
(24) Wu, L.; Courtney, K. C.; Chapman, E. R. Cholesterol Stabilizes Recombinant Exocytic Fusion Pores by Altering Membrane Bending Rigidity. Biophys. J. 2021, 120, 1367-1377.
(25) Studer, A.; Demarche, S.; Langenegger, D.; Tiefenauer, L. Integration and Recording of a Reconstituted Voltage-Gated Sodium Channel in Planar Lipid Bilayers. Biosens. Bioelectron. 2011, 26, 1924-1928.
(26) Su, C.-C.; Lyu, M.; Morgan, C. E.; Bolla, J. R.; Robinson, C. V; Yu, E. W. A ‘Build and Retrieve’ Methodology to Simultaneously Solve Cryo-EM Structures of Membrane Proteins. Nat. Methods 2021, 18, 69-75.
(27) Yao, X.; Fan, X.; Yan, N. Cryo-EM Analysis of a Membrane Protein Embedded in the Liposome. Proc. Natl. Acad. Sci. 2020, 117, 18497-18503.
(28) Aiyer, S.; Baldwin, P. R.; Tan, S. M.; Shan, Z.; Oh, J.; Mehrani, A.; Bowman, M. E.; Louie, G.; Passos, D. O.; Ðorević-Marquardt, S.; Mietzsch, M.; Hull, J. A.; Hoshika, S.; Barad, B. A.; Grotjahn, D. A.; McKenna, R.; Agbandje-McKenna, M.; Benner, S. A.; Noel, J. A. P.; Wang, D.; Tan, Y. Z.; Lyumkis, D. Overcoming Resolution Attenuation during Tilted Cryo-EM Data Collection. Nat. Commun. 2024, 15, 389.
(29) Tan, Y. Z.; Baldwin, P. R.; Davis, J. H.; Williamson, J. R.; Potter, C. S.; Carragher, B.; Lyumkis, D. Addressing Preferred Specimen Orientation in Single-Particle Cryo-EM through Tilting. Nat. Methods 2017, 14, 793-796.
(30) Punjani, A.; Rubinstein, J. L.; Fleet, D. J.; Brubaker, M. A. CryoSPARC: Algorithms for Rapid Unsupervised Cryo-EM Structure Determination. Nat. Methods 2017, 14, 290-296.
(31) Scheres, S. H. W. RELION: Implementation of a Bayesian Approach to Cryo-EM Structure Determination. J. Struct. Biol. 2012, 180, 519-530.
(32) Shi, L.; Cembran, A.; Gao, J.; Veglia, G. Tilt and Azimuthal Angles of a Transmembrane Peptide: A Comparison between Molecular Dynamics Calculations and Solid-State NMR Data of Sarcolipin in Lipid Membranes. Biophys. J. 2009, 96, 3648-3662.
(33) Shearer, J.; Jefferies, D.; Khalid, S. Outer Membrane Proteins OmpA, FhuA, OmpF, EstA, BtuB, and OmpX Have Unique Lipopolysaccharide Fingerprints. J. Chem. Theory Comput. 2019, 15, 2608-2619.
(34) Choi, Y. K.; Cao, Y.; Frank, M.; Woo, H.; Park, S.-J.; Yeom, M. S.; Croll, T. I.; Seok, C.; Im, W. Structure, Dynamics, Receptor Binding, and Antibody Binding of the Fully Glycosylated Full-Length SARS-COV-2 Spike Protein in a Viral Membrane. J. Chem. Theory Comput. 2021, 17, 2479-2487.
(35) Ni, T.; Frosio, T.; Mendonça, L.; Sheng, Y.; Clare, D.; Himes, B. A.; Zhang, P. High-Resolution in Situ Structure Determination by Cryo-Electron Tomography and Subtomogram Averaging Using EmClarity. Nat. Protoc. 2022, 17, 421-444.
(36) Fox, N. G.; Yu, X.; Feng, X.; Bailey, H. J.; Martelli, A.; Nabhan, J. F.; Strain-Damerell, C.; Bulawa, C.; Yue, W. W.; Han, S. Structure of the Human Frataxin-Bound Iron-Sulfur Cluster Assembly Complex Provides Insight into Its Activation Mechanism. Nat. Commun. 2019, 10, 2210.
(37) Ilitchev, A. How Pfizer is Driving Drug Discovery with Cryo-EM.
(38) Kampen, K. R. Membrane Proteins: The Key Players of a Cancer Cell. J. Membr. Biol. 2011, 242, 69-74.
(39) Maingi, V., Burns, J., Uusitalo, J. et al. Stability and dynamics of membrane-spanning DNA nanopores. Nat Commun 8, 14784 (2017).
(40) McAlduff D E, Heng Y M, Ottensmeyer F P. Freestanding lipid bilayers as substrates for electron cryomicroscopy of integral membrane proteins. J Microsc. 2002 February;205(Pt 2): 113-7.

1. A composition comprising:
(a) a substrate having a hydrophobic surface;
(b) a plurality of apertures through the substrate material; and
(c) a lipid bilayer extending across one or more of the plurality of apertures.

2. The composition of claim 1, wherein the substrate is a hydrophobic material selected from amorphous carbon film, pyrolytic carbon film, graphene, graphene oxide, and carbon nanotubes, polytetrafluoroethylene (PTFE [TEFLON]), polydimethylsiloxane, fluorinated ethylene propylene (FEP), fluorosilane, octadecyltrichlorosilane (OTS), polyethylene, and perfluoroalkoxy (PFA).

3. The composition of claim 1, wherein the substrate material is a chemically-modifiable material selected from gold, silicon nitride (Si₃N₄), silicon dioxide (SiO₂), titanium dioxide (TiO₂), aluminum oxide (Al₂O₃), zirconium oxide (ZrO₂), polyethylene (PE), polydimethylsiloxane (PDMS), a fluoropolymer (e.g., PTFE, FEP, PFA), polypropylene (PP), polytetrafluoroethylene (PTFE), and polyvinylidene fluoride (PVDF); and wherein the chemically-modifiable material has been modified to display a hydrophobic surface group selected from lipids, hydrocarbon chains, fatty acids, synthetic polymers, nanoparticles, short-chain perfluorocarbons, hydrophobic amino acids, and polyethylene glycol.

4. The composition of claim 1, wherein the substrate has a thickness of 1-100 nm, and optionally tapers at the edge of the apertures.

5. The composition of claim 1, wherein the plurality of apertures have diameters between 50 nm and 25 μm and are separated from each other by 50 nm to 20 μm.

6. The composition of claim 1, wherein the lipid bilayer comprises one or more components selected from glycerophosphlipids, sphingolipids, glycolipids, sterols, fatty acids, diacylglycerol, ether lipids, natural or synthetic lipidome, cell membrane extracted lipids mix, lipids with modified chains and/or headgroups, and fluorescent lipids, PEGylated lipids and any chemical modifications on these components.

7. The composition of claim 6, wherein the lipid bilayer further comprises one or more molecular or macromolecular structures of interest selected from membrane proteins, membrane embedded/associated particles, hydrophobic DNA, DNA nanostructures, peptides, polymers and any complex formed by these components with or without soluble proteins or DNA or nanoparticles or biomolecules or ligands or drugs.

8. The composition of claim 1, further comprising a mesh support beneath the substrate material; wherein the mesh support comprises a material selected from copper, gold, nickel, rhodium, molybdenum, silicon, carbon, and metal alloys or any metal; and wherein the mesh support comprises a plurality of interwoven or crossed metallic bands with gaps between them, where the bands are 1-200 μm in width and the gaps are 25-400 μm wide and the mesh support has an outer rim where the rim width is 50-800 μm

9. The composition of claim 1, wherein the lipid bilayer encompasses the substrate material at the edge of the one or more apertures.

10. A method of preparing a composition of claim 1, comprising:
(a) combining (i) components of the lipid bilayer, (ii) one or more detergents, and (iii) the substrate; and
(b) removing the one or more detergents under conditions that allow for formation of the lipid bilayer across one or more of the plurality of apertures.

11. The method of claim 10, wherein the one or more detergents are removed over a period of time ranging from 5 minutes to 120 hours.

12. The method of claim 10, wherein (i) the components of the lipid bilayer, (ii) the one or more detergents, and (iii) the substrate are combined in a vessel that is permeable to the one or more detergents.

13. The method of claim 12, wherein the one or more detergents are removed by placing the vessel in a solution or mixture with a lower concentration of the one or more detergents or absence of one or more detergents than within the vessel, and allowing the detergents to dialyze out of the vessel.

14. The method of claim 10, wherein the one or more detergents are removed adding an adsorbing agent capable of adsorbing the one or more detergents; wherein the adsorbing agent comprises one or more of polystyrene, polyethylene glycol, polysorbate, polyvinyl alcohol, poly(ethyleneimine), sepharose, silica resins, ion exchange resins, activated charcoal, fullerene derivatives, cyclodextrins, silica gel, alumina, and bovine serum albumin.

15. The method of claim 10, further comprising a step before step (b) of adding one or more molecular or macromolecular structures of interest selected from membrane proteins, membrane embedded/associated particles, hydrophobic DNA, DNA nanostructures, peptides, soluble proteins and polymers.

16. The method of claim 10, further comprising a step after step (b) of contacting the composition with one or more molecular or macromolecular structures of interest within a lipid-containing complex or solution and allowing the one or more molecular or macromolecular structures of interest to transfer from the lipid-containing complex or in solution to the lipid bilayer with or without membrane embedded/bound macromolecules/molecules of interest extending across one or more of the plurality of apertures.

17. The method of claim 16, wherein the lipid-containing complex is selected from nanodiscs, liposomes, micelles, lipid rafts, lipoplexes, lipid-polymer complexes, and proteoliposomes.

18. A method of preparing a composition of claim 1, comprising:
(a) applying lipids in an organic solvent to the substrate;
(b) drying the organic solvent;
(c) adding an aqueous buffer solution, thereby establishing a lipid bilayer extending across one or more of the plurality of apertures a lipid bilayer extending across one or more of the plurality of apertures.

19. The method of claim 18, further comprising contacting the composition with one or more molecular or macromolecular structures of interest within a lipid-containing complex and allowing the one or more molecular or macromolecular structures of interest to transfer from the lipid-containing complex to the lipid bilayer extending across one or more of the plurality of apertures.

20. A method of analyzing the structure of a molecular or macromolecular structures of interest embedded and/or associated with the lipid bilayer of the composition of claim 1, the method comprising conducting cryoEM (or another biophysical/analytical technique herein) analysis lipid bilayer extending across one or more of the plurality of apertures.
